(19) United States
(12) Patent Application Publication
(10) Pub. No.: US 2002/0132836 A1
(43) Pub. Date: Sep. 19, 2002

(54) Compounds and methods for modulating CCR4 function

(75) Inventors: Daniel J. Dairaghi, Palo Alto, CA (US); Brian E. McMaster, Mountain View, CA (US); Thomas J. Schall, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx Inc., San Carlos, CA (US)

(21) Appl. No.: 09/975,567

(22) Filed: Oct. 11, 2001

Related U.S. Application Data
(60) Provisional application No. 60/240,022, filed on Oct. 11, 2000.

(51) Int. Cl. A61K 31/426; A61K 31/4245; A61K 31/381; A61K 31/357; A61K 31/341; A61K 31/18

(52) U.S. Cl. 514/364; 514/369; 514/445; 514/452; 514/471; 514/602

(57) ABSTRACT

Compounds and compositions are provided that bind to the CCR4 chemokine receptor and which are useful for treating diseases associated with CCR4 activity, such as contact hypersensitivity.

### CROSS-REFERENCES TO RELATED APPLICATIONS

This application claims the benefit of Provisional Application Ser. No. 60/240,022, filed Oct. 11, 2000, the disclosure of which is incorporated herein by reference.

### STATEMENT AS TO RIGHTS TO INVENTIONS MADE UNDER FEDERALLY SPONSORED RESEARCH AND DEVELOPMENT

Not applicable

### BACKGROUND OF THE INVENTION

Chemokines are chemotactic cytokines that are released by a wide variety of cells to attract macrophages, T cells, eosinophils, basophils and neutrophils to sites of inflammation (reviewed in Schall, Cytokine, 3:165-183 (1991), Schall, et al., Curr. Opin. Immunol. 6:865-873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions ([Ca²⁺])i, granule exocytosis, integrin upregulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

There are two classes of chemokines, CXC (α) and CC (β), depending on whether the first two cysteines are separated by a single amino acid (C-X-C) or are adjacent (C-C). The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381:661-666 (1996)).

The chemokines bind specific cell-surface receptors belonging to the family of G-protein-coupled seven-transmembrane-domain proteins (reviewed in Horuk, Trends Pharm. Sci., 15:159-165 (1994)) which are termed “chemokine receptors.” On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC-CKR-1”) MIP-1α, MIP-1β, MCP-3, RANTES (Ben-Barruch, et al., J. Biol. Chem., 270, 22123-22128 (1995); Beote, et al, Cell, 72, 415-425 (1993)); CCR-2A and CCR-2B (or “CKR-2A”/“CKR-2A” or “CC-CKR-2A”/“CC-CKR2A”) MCP-1, MCP-3, MCP-4; CCR-3 (or “CKR-3” or “CC-CKR-3”) eotaxin, RANTES, MCP-3 (Combadiere, et al., J. Biol. Chem., 270, 16491-16494 (1995); CCR-4 (or “CKR-4” or “CC-CKR-4”) MIP-1α, RANTES, MCP-1 (Power, et al., J. Biol. Chem., 270, 19495-19500 (1995)); CCR-5 (or “CKR-5” or “CC-CKR-5”) MIP-1α, RANTES, MIP-1β (Sanson, et al., Biochemistry, 35, 3362-3367 (1996)); and the Duffy blood-group antigen RANTES, MCP-1 (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”).

Chemokine receptors, such as CCR1, CCR2, CCR2A, CCR2B, CCR3, CCR4, CCR5, CXCR3, CXCR4, have been implicated as being important mediators of inflammatory and immunoregulatory disorders and diseases, including asthma and allergic diseases, as well as autoimmune pathologies such as rheumatoid arthritis and atherosclerosis.

CC Chemokine Receptor 4, CCR4, is a G protein-coupled receptor that binds to chemokines including Macrophage-Derived Chemokine (MDC; a CC chemokine reported to be a chemoattractant for the Th2 subset of peripheral blood T cells, dendritic cells, and natural killer [NK] cells), and TARC (thymus and activation-regulated chemokine), which is also produced by monocytes and dendritic cells. To date, CCR4 has been reported to have restricted reactivity, in that it reacts with just MDC and TARC.

While the global distribution of CCR4 is unknown, the receptor is expressed on approximately 20% of adult peripheral blood effector/memory CD4+ T cells. Recent information suggests that CCR4 is present on almost all T cells that have a skin homing phenotype, the CTLA+ T cells. Thus CCR4 may be an important player in skin pathologies in which leukocytes participate. It also seems likely that CCR4 is expressed on some other cell types, possibly monocytes/macrophages and dendritic cells, among others.

In view of the clinical importance of CCR4, the identification of compounds that modulate CCR4 function represent an attractive avenue into the development of new therapeutic agents. Such compounds and methods for their use are provided herein.

### SUMMARY OF THE INVENTION

The present invention is directed to compositions and methods useful in modulating CCR4 chemokine receptor activity. The compounds, compositions and methods described herein are useful in the prevention or treatment of certain inflammatory and immunoregulatory disorders and diseases, including asthma and allergic diseases, as well as autoimmune pathologies such as rheumatoid arthritis and atherosclerosis.

More particularly, the compositions and methods provided herein use compounds having the general formula:

Ar¹—X—Ar² (I)

in which the symbols Ar¹ and Ar² each independently represent a substituted or unsubstituted aryl, substituted or unsubstituted heteroaryl, or a substituted or unsubstituted fused aryl-heterocyclic ring system. The letter X represents a linking group selected from —N(R)—, —C(O)S—, —CH═CHSO₂— and —SO₂N(R)— wherein R is H or a substituted or unsubstituted (C₁-C₈)alkyl group. In this formula, the linking group can be in either orientation relative to the Ar¹ and Ar² moieties. For example, the above general formula is meant to include both Ar¹—SO₂—NH—Ar² and Ar¹—NH—SO₂—Ar².

### BRIEF DESCRIPTION OF THE DRAWINGS

FIG. 1 is a graph showing the results of a CCR4/TARC competition assay using compound 1.1.

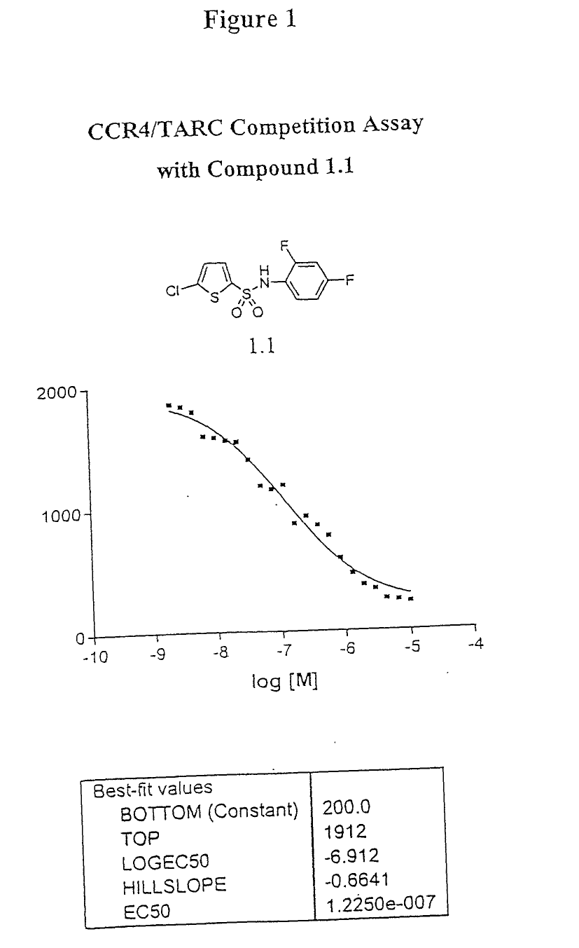

FIG. 2 is a graph showing the results of a CCR4/TARC competition assay using compound 1.2.

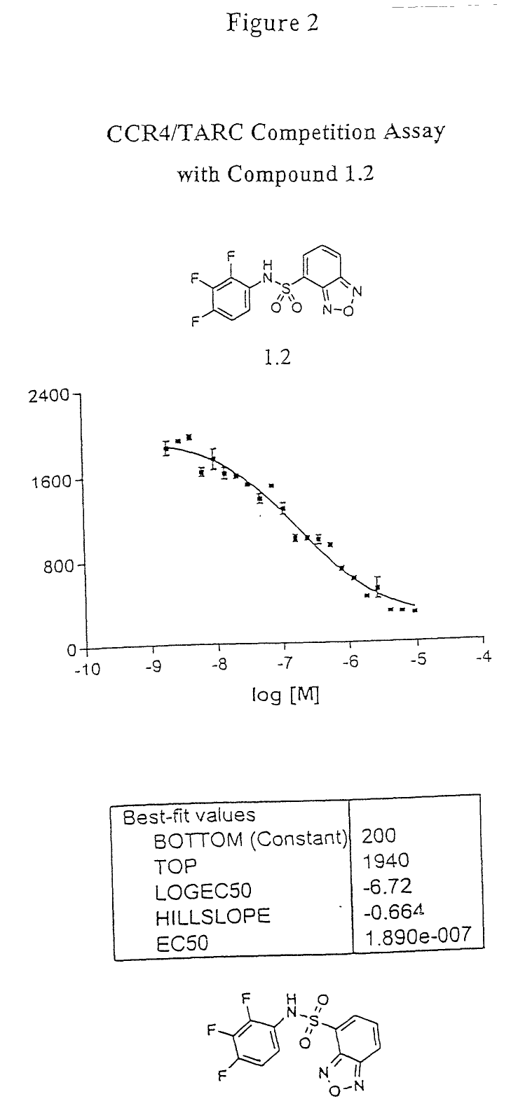

FIG. 3 is a graph showing the results of a CCR4/TARC competition assay using compound 1.3.

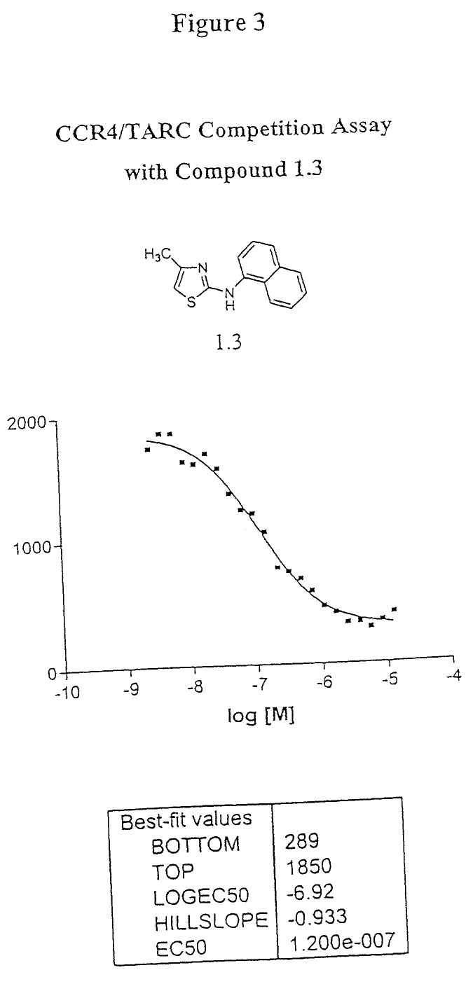

FIG. 4 illustrates the results achieved with compound 1.2 in CEM migration assays with CCR4 and TARC.

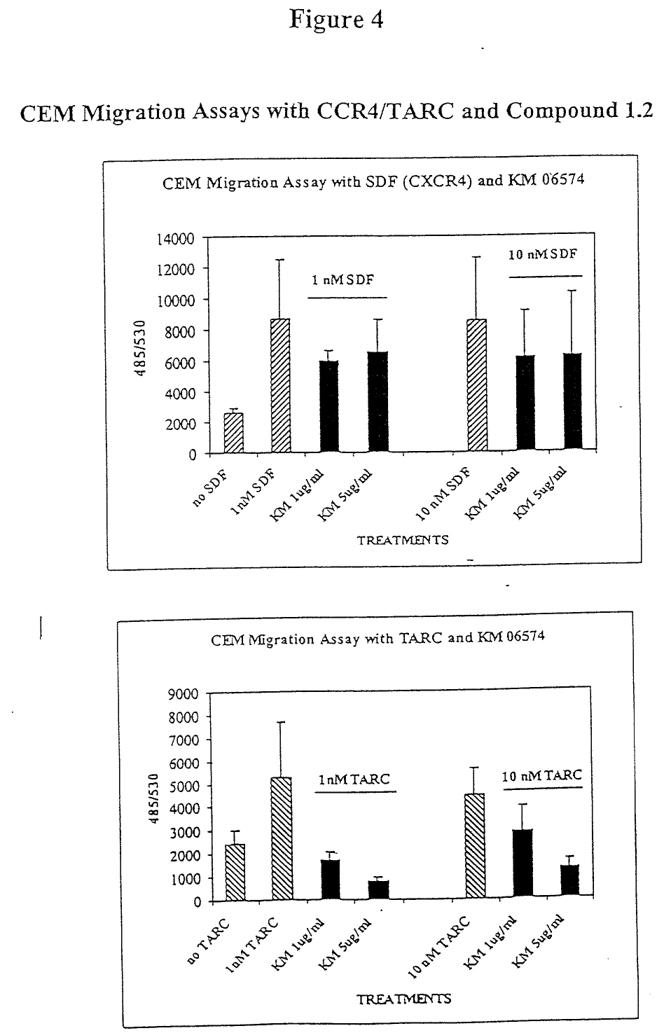

FIG. 5 illustrates the effect of compound 1.3 on CEM calcium response.

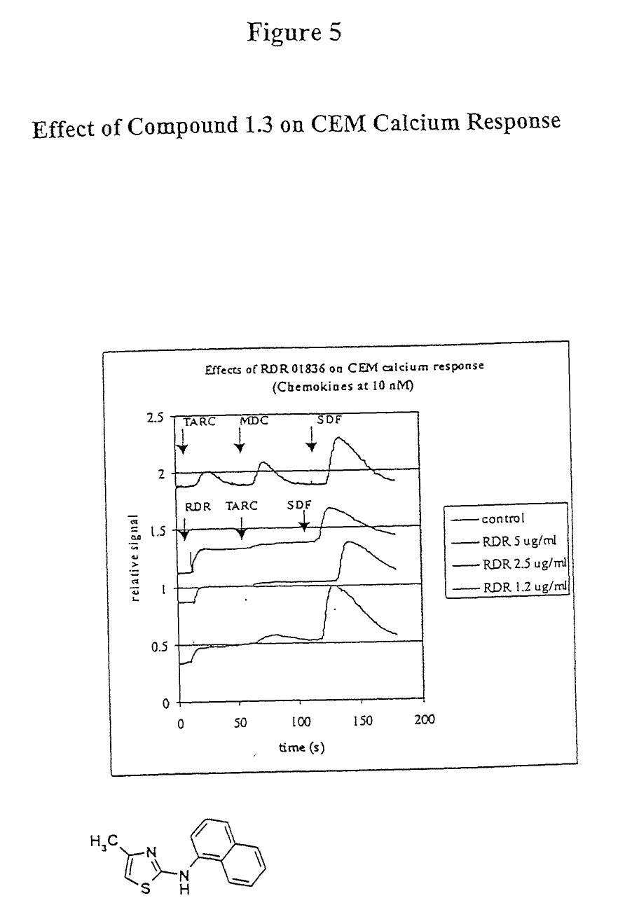

FIG. 6 illustrates the effect of compound 1.2 on CEM calcium response.

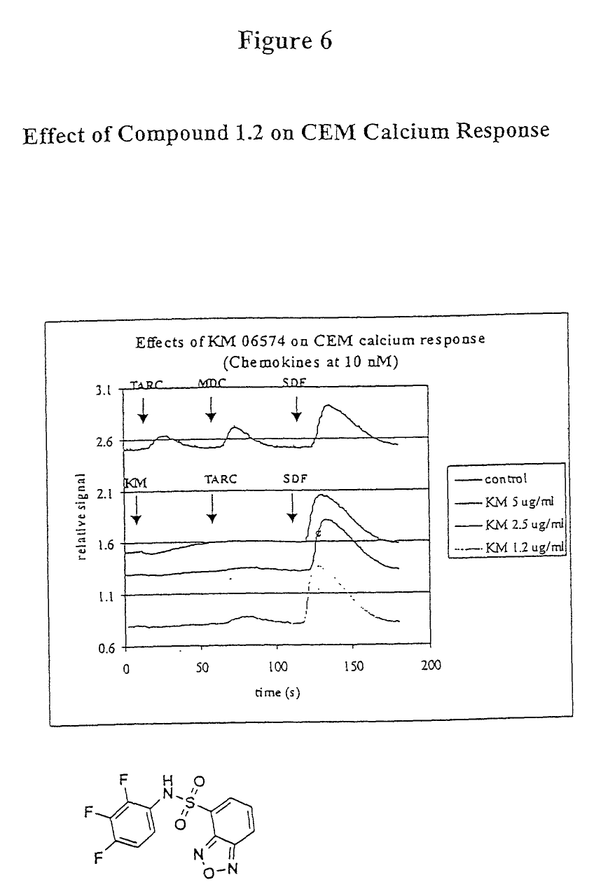

### DESCRIPTION OF THE INVENTION

Abbreviations and Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups which are limited to hydrocarbon groups are termed “homoalkyl”.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)— CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1 -(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula —T—C(O) —(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula —A—(CH₂)ᵣ—B—, wherein A and B are independently —CH₂—, —O—, —NH—, —S—, —S(O) —, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)2NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-25 (¹²⁵I) or carbon-14 (¹⁴C). All isotopic variations of the compounds of the present invention, whether radioactive or not, are intended to be encompassed within the scope of the present invention.

General

The present invention is directed to compounds, compositions and methods useful in the modulation of chemokine receptor activity, particularly CCR4. Accordingly, the compounds of the present invention are those which inhibit at least one function or characteristic of a mammalian CCR4 protein, for example, a human CCR4 protein. The ability of a compound to inhibit such a function can be demonstrated in a binding assay (e.g., ligand binding or promotor binding), a signalling assay (e.g., activation of a mammalian G protein, induction of rapid and transient increase in the concentration of cytosolic free calcium), and/or cellular response function (e.g., stimulation of chemotaxis, exocytosis or inflammatory mediator release by leukocytes).

Compositions that Modulate CCR4 Activity

In one aspect, the present invention provides compositions that modulate CCR4 activity. Generally, the compositions will comprise a pharmaceutically acceptable excipient and a compound having the general formula:

Ar¹—X—Ar² (I)

In formula I, the symbols Ar¹ and Ar² each independently represent a substituted or unsubstituted aryl, substituted or unsubstituted heteroaryl, or a substituted or unsubstituted fused aryl-heterocyclic ring system. The letter X represents a linking group selected from the group consisting of —N(R)—, —C(O)S—, —CH═CHSO₂— and —SO₂N(R)— wherein R is H or a substituted or unsubstituted (C₁-C₈)alkyl group. The linking group can be in either orientation relative to the Ar¹ and Ar² moieties. For example, the above general formula is meant to include both Ar¹—SO₂—NH—Ar² and Ar¹—NH—SO₂—Ar².

Returning to formula I above, in one group of preferred embodiments, Ar¹ is a substituted or unsubstituted heteroaryl group. Preferably Ar¹ is a substituted or unsubstituted 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, or 6-quinolyl group. More preferably, Ar¹ is a substituted or unsubstituted thiazolyl, thienyl, benzoxadiazolyl or oxazolyl group.

In another group of preferred embodiments, Ar² is a substituted or unsubstituted aryl group. More preferably, Ar² is a substituted or unsubstituted phenyl or naphthyl group. For those embodiments in which Ar² is a substituted aryl group, there will preferably be from 1 to 4 substituents, independently selected from halogen, hydroxy, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino. More preferably, there will be 1 to 3 substituents independently selected from halogen, (C₁-C₄)haloalkyl, (C₁-C₄)haloalkoxy, nitro, cyano and (C₁-C₄)acyl. In the most preferred embodiments, Ar² is a substituted or unsubstituted phenyl or naphthyl group. The substituted phenyl and napthyl groups will preferably have from 1 to 3 substituents independently selected from halogen, (C₁-C₄)haloalkyl, (C₁-C₄)haloalkoxy, nitro, cyano and (C₁-C₄)acyl.

In another group of preferred embodiments, Ar¹ and Ar² are each members independently selected from the group consisting of:

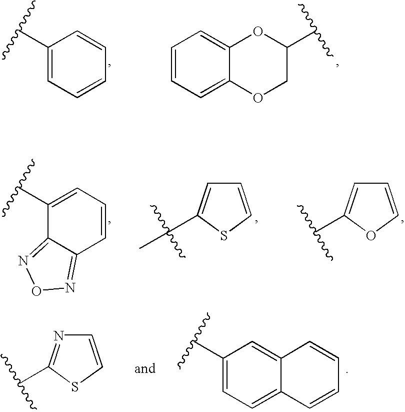

Further preferred are those embodiments in which Ar¹ is selected from substituted or unsubstituted phenyl or 2-naphthyl and Ar¹ is selected from substituted or unsubstituted

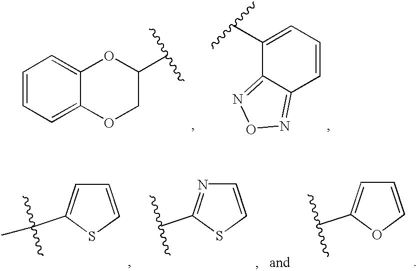

In the most preferred embodiments, the compounds used in the present compositions are selected from:

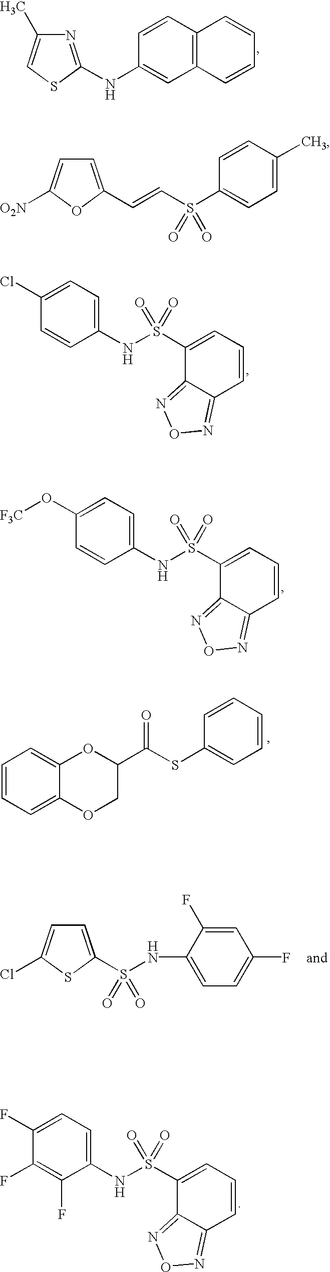

In addition to the compounds provided above, compositions for modulating chemokine receptor activity in humans and animals will typically contain a pharmaceutical carrier or diluent.

“Modulation” of chemokine receptor activity, as used herein in its various forms, is intended to encompass antagonism, agonism, partial antagonism and/or partial agonism of the activity associated with a particular chemokine receptor, preferably the CCR4 receptor. The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

The pharmaceutical composition and method of the present invention may further comprise other therapeutically active compounds as noted herein which are usually applied in the treatment of the above mentioned pathological conditions.

Methods of Treating CCR4-Mediated Conditions or Diseases

In yet another aspect, the present invention provides methods of treating CCR4-mediated conditions or diseases by administering to a subject having such a disease or condition, a therapeutically effective amount of a compound of formula I above. The “subject” is defined herein to include animals such as mammals, including, but not limited to, primates (e.g., humans), cows, sheep, goats, horses, dogs, cats, rabbits, rats, mice and the like.

The term “therapeutically effective amount” means the amount of the subject compound that will elicit the biological or medical response of a tissue, system, animal or human that is being sought by the researcher, veterinarian, medical doctor or other clinician.

Diseases and conditions mediated by CCR4 include contact hypersensitivity, atopic dermatitis, allergic airway hypersensitivity, atherosclerosis, diseases of innate immunity (e.g., septic shock), and diseases or conditions caused by platelet aggregation or thrombosis. More specifically, diseases or conditions mediated by CCR4 include, but are not limited to, contact hypersensitivity, atopic dermatitis and other skin diseases (e.g., psoriasis), asthma, allergic rhinitis, atherosclerosis, septic shock, angina, myocardial infarction, restenosis, ischemia/reperfusion injury, and stroke. Still other diseases mediated by CCR4 include inflammation, infection and cancer. More specifically, these diseases or conditions include: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; hypersensitivity lung diseases, and the like, (2) autoimmune diseases, such as arthritis (rheumatoid and psoriatic), multiple sclerosis, systemic lupus erythematosus, diabetes, glomerulonephritis, and the like, (3) graft rejection (including allograft rejection and graft-v-host disease), and (4) other diseases in which undesired inflammatory responses are to be inhibited (e.g., atherosclerosis, myositis).

Depending on the disease to be treated and the subject's condition, the compounds of the present invention may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the present invention can be combined with other compounds having related utilities to prevent and treat inflammatory and immunoregulatory disorders and diseases, including asthma and allergic diseases, as well as autoimmune pathologies such as rheumatoid arthritis and atherosclerosis, and those pathologies noted above.

For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists, (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3 and CCR-5; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), .alpha.-glucosidase inhibitors (acarbose) and glitazones (troglitazone and pioglitazone); (1) preparations of interferon beta (interferon beta- .alpha., interferon beta-i.beta.); (m) other compounds such as 5-aminosalicylic acid and prodrugs thereof, antimetabolites such as azathioprine and 6-mercaptopurine, and cytotoxic cancer chemotherapeutic agents. The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1: 1000, preferably about 200:1 to about 1:200. Combinations of a compound of the present invention and other active ingredients will generally also be within the aforementioned range, but in each case, an effective dose of each active ingredient should be used.

Method of Evaluating Putative CCR4 Modulators

In yet another aspect, the present invention includes methods to evaluate putative specific agonists or antagonists of CCR4 function. Accordingly, the present invention is directed to the use of these compounds in the preparation and execution of screening assays for compounds which modulate the activity of the CCR4 chemokine receptor. For example, the compounds of this invention are useful for isolating receptor mutants, which are excellent screening tools for more potent compounds. Furthermore, the compounds of this invention are useful in establishing or determining the binding site of other compounds to the CCR4 chemokine receptor, e.g., by competitive inhibition. The compounds of the instant invention are also useful for the evaluation of putative specific modulators of the CCR4 chemokine receptor, relative to other chemokine receptors including CCR-1, CCR-2, CCR-2A, CCR-2B, CCR-3, CCR-5, CXCR-3 and CXCR-4. One of skill in the art will appreciate that thorough evaluation of specific agonists and antagonists of the above chemokine receptors has been hampered by the lack of availability of non-peptidyl (metabolically resistant) compounds with high binding affinity for these receptors. Thus the compounds provided herein are particularly useful in this context.

Preparation of CCR4 Modulators

A number of compounds useful as modulators of CCR4 signalling can be obtained from commercial sources such as Maybridge Chemical Co. and Aldrich Chemical Co. (Milwaukee, Wis., USA).

Other compounds can be prepared using conventional methods. For example, Scheme 1 illustrates the preparation of diarylsulfonamides.

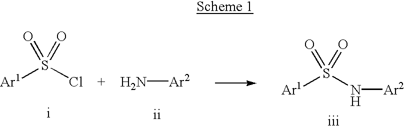

In this scheme, an aryl (or heteroaryl) sulfonyl chloride i can be combined with a suitable aryl (or heteroaryl) amine ii to provide the target sulfonamides iii. The starting sulfonyl chlorides are typically prepared in one step from a sulfonic acid using a chlorinating agent such as POC1₃ or SOC1₂. Similarly, starting arylamines ii are available from commercial sources or can be prepared in one step from the corresponding nitroaryl compounds.

Evaluation Assays for CCR4 Modulators

A variety of assays can be used to evaluate the compounds provided herein, including CCR4 binding assays, CCR4 signalling assays, chemotaxis assays, and other assays of cellular response.

In a suitable assay, a CCR4 protein (whether isolated or recombinant) is used which has at least one property, activity or functional charateristic of a mammalian CCR4 protein. The property can be a binding property (to, for example, a ligand or inhibitor), a signalling activity (e.g., activation of a mammalian G protein, induction of rapid and transient increase in the concentration of cytosolic free calcium [Ca⁺⁺]ᵢ), cellular response function (e.g., stimulation of chemotaxis or inflammatory mediator release by leukocytes), and the like.

In one embodiment, a composition containing a CCR4 protein or variant thereof is maintained under conditions suitable for binding. The CCR4 receptor is contacted with a putative agent (or a second composition containing at least one putative agent) to be tested, and binding is detected or measured.

In one group of preferred embodiments, the assay is a cell-based assay and cells are used which are stably or transiently transfected with a vector or expression cassette having a nucleic acid sequence which encodes the CCR4 receptor. The cells are maintained under conditions appropriate for expression of the receptor and are contacted with a putative agent under conditions appropriate for binding to occur. Binding can be detected using standard techniques. For example, the extent of binding can be determined relative to a suitable control (for example, relative to background in the absence of a putative agent, or relative to a known ligand). Optionally, a cellular fraction, such as a membrane fraction, containing the receptor can be used in lieu of whole cells.

Detection of binding or complex formation can be detected directly or indirectly. For example, the putative agent can be labeled with a suitable label (e.g., fluorescent label, chemiluminescent label, isotope label, enzyme label, and the like) and binding can be determined by detection of the label. Specific and/or competitive binding can be assessed by competition or displacement studies, using unlabeled agent or a ligand (e.g., TARC or MDC) as a competitor.

In other embodiments, binding inhibition assays can be used to evaluate the present compounds. In these assays, the compounds are evaluated as inhibitors of ligand binding using, for example, TARC or MDC. In this embodiment, the CCR4 receptor is contacted with a ligand such as TARC or MDC and a measure of ligand binding is made. The receptor is then contacted with a test agent in the presence of a ligand (e.g., TARC or MDC) and a second measurement of binding is made. A reduction in the extent of ligand binding is indicative of inhibition of binding by the test agent. The binding inhibition assays can be carried out using whole cells which express CCR4, or a membrane fraction from cells which express CCR4.

The binding of a G protein-coupled receptor by, for example, an agonist, can result in a signalling event by the receptor. Accordingly, signalling assays can also be used to evaluate the compounds of the present invention and induction of signalling function by an agent can be conitored using any suitable method. For example, G protein activity, such as hydrolysis of GTP to GDP, or later signalling events triggered by receptor binding can be assayed by known methods (see, for example, PCT/US97/15915; Neote, et al., Cell, 72:415-425 (1993); Van Riper, et al., J. Exp. Med., 177:851-856 (1993) and Dahinden, et al., J. Exp. Med., 179:751-756 (1994)).

Chemotaxis assays can also be used to assess receptor function and evaluate the compounds provided herein. These assays are based on the functional migration of cells in vitro or in vivo induced by an agent, and can be used to assess the binding and/or effect on chemotaxis of ligands, inhibitors, or agonists. Suitable assays are described in PCT/US97/15915; Springer, et al., WO 94/20142; Berman et al., Immunol Invest., 17:625-677 (1988); and Kavanaugh et al., J. Immunol., 146:4149-4156 (1991)).

The compounds provided herein can also be evaluated using models of inflammation to assess the ability of the compound to exert an effect in vivo. Suitable models are described as follows: a sheep model for asthma (see, Weg, et al., J. Exp. Med., 177:561 (1993)); and a rat delayed-type hypersensitivity model (see Rand, et al., Am. J. Pathol., 148:855-864 (1996)). Another useful model for evaluating the instant compounds is the experimental autoimmune encephalomyelitis (EAE) model for multiple sclerosis, which probes chemokine receptor expression and function (see, Ransohoff, et al., Cytokine Growth Factor Rev., 7:35-46 (1996), and Karpus, et al., J Immunol. 161:2667-2671 (1998)).

In addition, leukocyte infiltration assays can also be used to evaluate a compound (see, Van Damme, et al., J. Exp. Med. 176:59-65 (1992); Zachariae, et al., J. Exp. Med. 171:2177-2182 (1990); and Jose, et al., J. Exp. Med. 179:881-887 (1994)).

### EXAMPLES

The following examples are offered to illustrate, but not to limit the claimed invention.

### Example 1

This example illustrates screening procedures used in characterizing the compounds of the present invention.

Source plates of chemical libraries were obtained from commercial vendors and contained individual compounds at 5 mg/mL in DMSO, or in some instances, at 1 mg/mL. From these, multiple compound plates containing 10 compounds in each well were made, and these were diluted in 20% DMSO to a concentration of 50 μg/mL (10 μg/mL for those beginning at 1 mg/mL). An aliquot of 20 μL of each mixture was put into the test plates, which were stored frozen until use.

A CCR4 expressing stable transfectant cell line was prepared using current standard molecular biological methods. The CCR4 transfectants were cultured in IMDM-5% FBS, and harvested when the concentration was between 0.5-1.0×10⁶ cells/ml. The cells were centrifuged and resuspended in assay buffer (20 mM HEPES pH 7.1, 140 mM NaCl, 1 mM CaCl₂, 5 mM MgCl₂, and with 0.2% bovine serum albumin) to a concentration of 5.6×10⁶ cells/ml. To set up the screening assays, first 0.09 ml of cells was added to the assay plates containing the compounds. (For a final compound concentration of 1-5 1g/ml each [2-10 μM]). Then 0.09 ml of ¹²⁵I labeled MDC or TARC diluted in assay buffer (final concentration ˜50 pM, with ˜30,000 cpm per well) was added. The plates were sealed and incubated for approximately 3 hours at 4° C. on a shaker platform. The assay plates were harvested using Packard filter plates, pre-soaked in 0.3% PEI (polyethyleneimine) solution, on a Packard vacuum cell harvester. Scintillation fluid (50 μl) was added to all wells and the plates were sealed and counted in a Top Count scintillation counter. Control wells containing either diluent only (for total counts) or excess MDC or TARC (1 μg/ml, for non-specific binding) were used to calculate the percent of total inhibition for each set of compounds. Further tests on individual compounds were carried out in the same manner. IC₅₀ values are those concentrations required to reduce the binding of labeled MDC or TARC to the receptor by 50%.

The calcium mobilization experiments were performed by labeling the human T-cell line CEM with INDO-1 dye (45 min at room temperature), washing with PBS, and resuspending into flux buffer (HBSS with 1% fetal bovine serum). For each test, 1×10⁶ cells were incubated at 37° C. in the cuvette of a PTI spectrometer, and the ratio of 410/490 nm emission plotted over time (typically 2-3 minutes), with compounds added at 5 seconds, followed by MDC, TARC or other chemokines.

Chemotaxis assays were performed using 5μ filter plates (Neuroprobe) with the chemoattractant (MDC, TARC, or SDF) placed in the lower chamber, and a cell suspension of 100,000 CEM cells in the upper chamber. The assays were incubated 1-2 hours at 37° C., and the number of cells in the lower chamber quantified by the CyQuant assay (Molecular Probes).

Results are provided in Figures.

### Example 2

This example describes a procedure to evaluate the efficacy of CCR4 antagonists for treatment of septic shock.

An animal model of endotoxic shock can be induced by injecting rodents with lipopolysaccharide (LPS). Three series of mouse groups, comprising 15 mice per group, are treated with an intra-peritoneal injection of an L.D.(lethal dose)-90 of LPS (precise dose requires titration of the particular batch of lipopolysaccharide in the actual mouse colony in use to determine a re-producible dose of LPS that produces 90% mortality in mice). One series of mice additionally receives phosphate buffered saline (PBS) and Tween 0.5% i.p. 30 minutes before LPS administration. A second series consists of groups of mice receiving different doses of the CCR4 antagonist(s) given either intra-peritoneally, intra-venously, sub-cutaneously, intramuscularly, orally, or via any other mode of administration 30 minutes before, or concurrently with, LPS administration. A third series of mice, serving as positive control, consists of groups treated with either mouse IL-10 i.p., or anti-TNF antibodies i.p., 30 minutes before LPS administration.

Mice are monitored for death for 72 hours following the LPS injection.

### Example 3

This example describes a procedure to evaluate the efficacy of CCR4 antagonists for treatment of asthma.

An animal model of asthma can be induced by sensitizing rodents to an experimental antigen (e.g. OVA) by standard immunization, and then subsequently introducing that same antigen into the rodents lung by aerosolization. Three series of rodent groups, comprising 10 rodents per goup, are actively sensitized on Day 0 by a single intraperitoneal injection with 100 ug OVA in phosphate-buffered saline (PBS), along with an IgE-selective adjuvant e.g. aluminum hydroxide. At 11 days after sensitization, at the peak of their IgE response, the animals are placed in a Plexiglas chamber and challenged with aerosolized OVA (1%) for 30 minutes using the ultrasonic nebulizer (De Vilbliss). One series of mice additionally receives phosphate buffered saline (PBS)and Tween 0.5% i.p. at the initial sensitization, and at different dosing schedules thereafter, up until the aerosolized OVA challenge. A second series consists of groups of mice receiving different doses of the CCR4 antagonist(s) given either intra-peritoneally, intra-venously, sub-cutaneously, intramuscularly, orally, or via any other mode of administration at the initial sensitization, and at different dosing schedules thereafter, up until the aerosolized OVA challenge. A third series of mice, serving as positive control, consists of groups treated with either mouse IL-10 i.p., anti-IL4 antibodies i.p., or anti-IL5 antibodies i.p. at the initial sensitization, and at different dosing schedules thereafter, up until the aerosolized OVA challenge.

Animals were subsequently analyzed at different time points after the aerosolized OVA challenge for pulmonary function, cellular infiltrates in bronchoalveolar lavage (BAL), histological examination of lungs, and measurement of serum OVA-specific IgE titers.

### Example 4

This example describes a procedure to evaluate the efficacy of CCR4 antagonists for augmenting protective immunity against viruses, bacteria and parasites.

Protective immunity to microbial pathogens is frequently mediated by Th1 regulatory T cells. Since CCR4 antagonists are likely inhibitors of Th2 regulatory cells, they may alter the cross regulation that normally exists between Thl and Th2 cells, and potentiate Th1 cells, thereby augmenting protection against infectious disease. Three series of mouse groups, comprising 15 mice per group, are infected with the intracellular parasite Leishmania major (L.major) by injecting L.major promastigotes sub-cutaneously into their left hind footpads. Four weeks after infection, the animals are challenged with either Leishmania freeze-thawed antigen, or PBS as a negative control, in the contra-lateral footpad. One series of mice additionally receives phosphate buffered saline (PBS)and Tween 0.5% i.p. at the initial sensitization, and at different dosing schedules thereafter, up until the Leishmania antigen challenge. A second series consists of groups of mice receiving different doses of the CCR4 antagonist(s) given either intra-peritoneally, intra-venously, sub-cutaneously, intramuscularly, orally, or via any other mode of administration at the initial sensitization, and at different dosing schedules thereafter, up until the Leishmania antigen challenge. A third series of mice, serving as positive control, consists of groups treated with either mice IL-12, anti-IL4 antibodies i.p., or anti-IL5 antibodies i.p. at the initial sensitization, and at different dosing schedules thereafter, up until the Leishmania antigen challenge.

Over the next 48 hours, footpad swelling, caused by a Delayed-Type Hypersensitivity reaction to the Leishmania antigen challenge, is monitored with a metric caliper. The response of draining lymph node T cells to Leishmania antigen stimulation in vitro is also measured, both at the level of proliferation, cytokine production, and other phenotypic criteria.

### Example 5

This example describes a procedure to evaluate the efficacy of CCR4 antagonists for treatment of rheumatoid arthritis.

An animal model of rheumatoid arthritis can be induced in rodents by injecting them with type II collagen in selected adjuvants. Three series of rodent groups consisting 15 genetically-susceptible mice or rats per group are injected sub-cutaneously or intra-dermally with type II collagen emulsified in Complete Freund's Adjuvant at days 0 and 21. One series of rodents additionally receives phosphate buffered saline (PBS) and Tween 0.5% i.p. at the initial sensitization, and at different dosing schedules thereafter. A second series consists of groups of rodents receiving different doses of the CCR4 antagonist(s) given either intra-peritoneally, intra-venously, sub-cutaneously, intra-muscularly, orally, or via any other mode of administration at the initial sensitization, and at different dosing schedules thereafter. A third series of rodents, serving as positive control, consists of groups treated with either mouse IL-10 i.p., or anti-TNF antibodies i.p.at the initial sensitization, and at different dosing schedules thereafter.

Animals are monitored from weeks 3 til 8 for the development of swollen joints or paws, and graded on a standard disease severity scale. Disease severity is confirmed by histological analysis ofjoints.

### Example 6

This example describes a procedure to evaluate efficacy of CCR4 antagonists for treatment of Systemic Lupus Erythematosus (SLE).

Female NZB/W F1 mice spontaneously develop an SLE-like pathology commencing at 6 months of age that is characterized by proteinuria, serum autoantibodies, glomerulonephritis, and eventually death. Three series of NZB/W mouse groups comprising 20 mice per group are tested for efficacy of CCR antagonist(s) as follows: One series of mice additionally receives phosphate buffered saline (PBS) and Tween 0.5% i.p. soon after weaning, and thereafter at varying dosing schedules. A second series consists of groups of mice receiving different doses of the CCR4 antagonist(s) given either intra-peritoneally, intra-venously, sub-cutaneously, intra-muscularly, orally, or via any other mode of administration soon after weaning, and thereafter at varying dosing schedules. A third series of mice, serving as positive control, consists of groups treated with anti-IL10 antibodies given soon after weaning, and thereafter at varying dosing schedules.

Disease development is monitored in terms of eventual mortality, kidney histology, serum autoantibody levels, and proteinuria.

### Example 7

This example describes a procedure to evaluate efficacy of CCR4 antagonists for treatment of malignancy.

SCID mice can be transplanted with primary human tumor cells. Normal mouse strains can be transplanted with a variety of well-characterized mouse tumor lines, including a mouse thymoma EL4 which has been transfected with OVA to allow easy evaluation of tumor specific antigen responses following vaccination with OVA. Three series of mouse groups from any of these tumor models are tested for CCR4 antagonist efficacy as follows: One series of mice additionally receives phosphate buffered saline (PBS) and Tween 0.5% i.p. soon after tumor transplant, and thereafter at varying dosing schedules. A second series consists of groups of mice receiving different doses of the CCR4 antagonist(s) given either intra-peritoneally, intra-venously, sub-cutaneously, intra-muscularly, orally, or via any other mode of administration soon after tumor transplant, and thereafter at varying dosing schedules. A third series of mice, serving as positive control, consists of groups treated with either anti-IL4 antibodies, anti-IFNg antibodies, IL4, or TNF, given i.p. soon after tumor transplant, and thereafter at varying dosing schedules.

Efficacy is monitored via tumor growth versus regression. In the case of the OVA-transfected EL4 thymoma model, cytolytic OVA-specific responses can be measured by stimulating draining lymph node cells with OVA in vitro, and measuring antigen-specific cytotoxicity at 72 hours.

### Example 8

This example describes procedures to evaluate the efficacy of CCR4 antagonists in psoriasis.

A rodent model of psoriasis can be obtained by intra-venously transferring a population of purified T cells (designated CD45Rbhi T cells) obtained from the spleens of BALB/c mice into immunodeficient recipient CB. 17 scid/scid mice. Mice develop signs of redness, swelling, and skin lesions resembling those of human psoriasis in their ear, feet and tail by 8 weeks after transfer. Three series of mouse groups, comprising 10-15 CB.17 scid/scid mice per group, are injected with purified CD45Rbhi T cells. One series of mice additionally receives phosphate buffered saline (PBS) and Tween 0.5% i.p. at the initial cell transfer, and at different dosing schedules thereafter. A second series consists of groups of mice receiving different doses of the CCR4 antagonist(s) given either intra-peritoneally, intra-venously, sub-cutaneously, intramuscularly, orally, or via any other mode of administration at the initial cell transfer, and at different dosing schedules thereafter. A third series of mice, serving as positive control, consists of groups treated with antibodies to either IL-12, IL-4, IFNg, or TNF, or with cytokine IL-10 at the initial cell transfer, and at different dosing schedules thereafter. Animals are monitored for development of psoriatic-like lesions for 3 months after cell transfer.

### Example 9

This example describes a procedure to evaluate the efficacy of CCR4 antagonists in Inflammatory Bowel Disease (IBD).

Several mouse models of IBD (including Crohn's Disease and Ulcerative Colitis) have been developed. Some of these are spontaneous models occurring in genetically engineered transgenic mice that have been depleted of certain cytokine genes (e.g. IL-10, or IL-2) by homologous recombination. Another mouse model of Inflammatory Bowel Disease is obtained by transferring highly purified populations of CD4+ T lymphocytes bearing a particular surface marker phenotype (namely CD45 RB hi) into SCID mice. Three series of mouse groups from any one of these models can be used to evaluate CCR4 antagonist efficacy as follows. One group of mice additionally receives phosphate buffered saline (PBS) and Tween 0.5% i.p. soon after weaning in the case of the spontaneous models in transgenic mice, or at time of cell transfer into SCID mice and varying dosings thereafter for the cell transfer model. A second series consists of groups of mice receiving different doses of the CCR4 antagonist(s) given either intra-peritoneally, intra-venously, sub-cutaneously, intramuscularly, orally, or via any other mode of administration soon after weaning in the case of the spontaneous models in transgenic mice, or at time of cell transfer into SCID mice and varying dosings thereafter for the cell transfer model. A third series of mice, serving as positive control, consists of groups treated with antibodies to either IFNg, or TNF, or with cytokine IL-10 soon after weaning in the case of the spontaneous models in transgenic mice, or at time of cell transfer into SCID mice and varying dosings thereafter for the cell transfer model.

Mice are evaluated for 6-8 weeks for disease development, monitored initially via weight loss and/or prolapsed rectum, and eventually by histological evaluation of the animals colon and intestinal tract.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating a CCR4-mediated condition or disease in a subject, said method comprising administering to a subject in need of such treatment an effective amount of a compound having the formula:
Ar¹′X—Ar² (I)
wherein
Ar¹ and Ar² are each members independently selected from the group consisting of substituted or unsubstituted aryl, substituted or unsubstituted fused aryl-heterocyclic ring systems and substituted or unsubstituted heteroaryl; and
X is a linking group selected from the group consisting of —N(R)—, —C(O)S—, —CH═CHSO₂— and —SO₂N(R)— wherein R is a member selected from the group consisting of H and substituted or unsubstituted (C₁-C₈)alkyl.

2. A method in accordance with claim 1, wherein X is —NH—.

3. A method in accordance with claim 1, wherein X is —SO₂NH—.

4. A method in accordance with claim 1, wherein Ar¹ and Ar² are each substituted or unsubstituted members independently selected from the group consisting of:

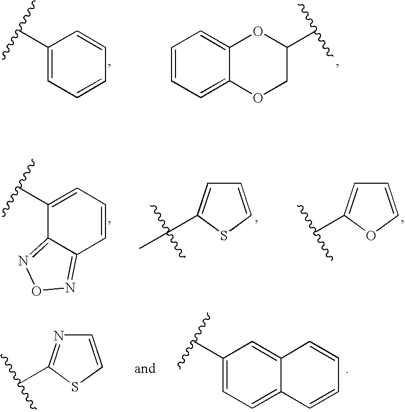

5. A method in accordance with claim 2, wherein Ar¹ is substituted heteroaryl and Ar² is substituted or unsubstituted aryl.

6. A method in accordance with claim 5, wherein said Ar¹ is a substituted heteroaryl selected from the group consisting of substituted thiazolyl, substituted thienyl, and substituted furanyl.

7. A method in accordance with claim 5, wherein said Ar² is a substituted or unsubstituted phenyl or a substituted or unsubstituted naphthyl.

8. A method in accordance with claim 3, wherein Ar² is a phenyl group having from 1 to 4 substituents independently selected from the group consisting of halogen, hydroxy, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino.

9. A method in accordance with claim 8, wherein said phenyl group has from 1 to 3 substituents independently selected from the group consisting of halogen, (C₁-C₄)haloalkyl, (C₁-C₄)haloalkoxy, nitro, cyano, and (C₁-C₄)acyl.

10. A method in accordance with claim 3, wherein Ar¹ is a substituted or unsubstituted monocyclic or bicyclic heterocycle.

11. A method in accordance with claim 10, wherein said heterocycle is selected from the group consisting of pyrrolyl, pyrazolyl, imidazolyl, pyrazinyl, oxazolyl, isoxazolyl, thiazolyl, furyl, thienyl, pyridyl, pyrimidyl, benzothiazolyl, benzoxadiazolyl, purinyl, benzimidazolyl, indolyl, isoquinolyl, quinoxalinyl and quinolyl.

12. A method in accordance with claim 11, wherein said heterocycle is selected from the group consisting of thienyl, thiazolyl and benzoxadiazolyl.

13. A method in accordance with claim 1, wherein said CCR4-mediated condition or disease is selected from the group consisting of contact hypersensitivity, atopic dermatitis, allergic airway hypersensitivity, allergic rhinitis, atherosclerosis, septic shock, angina, myocardial infarction, restenosis, ischemia/reperfusion injury, multiple sclerosis, rheumatoid arthritis, type I diabetes, psoriasis, cancer and HIV infection.

14. A method in accordance with claim 1, wherein said CCR4-mediated condition or disease is psoriasis, contact hypersensitivity or atopic dermatitis.

15. A method in accordance with claim 14, wherein said CCR4-mediated condition or disease is psoriasis.

16. A method in accordance with claim 14, wherein said CCR4-mediated condition or disease is contact hypersensitivity.

17. A method in accordance with claim 14, wherein said CCR4-mediated condition or disease is atopic dermatitis.

18. A method in accordance with claim 1, wherein said CCR4-mediated condition or disease is a disease of the airway.

19. A method in accordance with claim 18, wherein said disease of the airway is selected from the group consisting of allergic asthma and allergic rhinitis.

20. A method in accordance with claim 18, wherein said disease of the airway is allergic asthma.

21. A method in accordance with claim 1, wherein said CCR4-mediated condition or disease is a disease of innate immunity.

22. A method in accordance with claim 21, wherein said disease of innate immunity is septic shock.

23. A method in accordance with claim 1, wherein said CCR4-mediated condition or disease is atherosclerosis.

24. A method in accordance with claim 1, wherein said CCR4-mediated condition or disease is a disease or condition characterized by platelet aggregation or thrombosis.

25. A method in accordance with claim 24, wherein said CCR4-mediated disease or condition is selected from the group consisting of angina, myocardial infarction, restenosis, stroke and ischemia/reperfusion injury.

26. A method in accordance with claim 1, wherein said CCR4-mediated condition or disease is an allergic condition and said compound is used alone or in combination with at least one therapeutic agent wherein said therapeutic agent is an antihistamine.

27. A method in accordance with claim 1, wherein said CCR4-mediated disease or condition is psoriasis and said compound is used alone or in combination with at least one therapeutic agent selected from a corticosteroid, a lubricant, a keratolytic agent, a vitamin D₃ derivative, PUVA, or anthralin.

28. A method in accordance with claim 1, wherein said CCR4-mediated disease or condition is atopic dermatitis and said compound is used alone or in combination with at least one therapeutic agent selected from a lubricant and corticosteroid.

29. A method in accordance with claim 1, wherein said CCR4-mediated condition or disease is asthma and said compound is used alone or in combination with at least one therapeutic agent selected from a B2-agonist and a corticosteroid.

30. A method in accordance with claim 1, wherein said compound interferes with the interaction between CCR4 and a ligand.

31. A method in accordance with claim 1, wherein said administration is oral or intravenous.

32. A method in accordance with claim 1, wherein said subject is selected from the group consisting of human, rat, dog, cow, horse, and mouse.

33. A method in accordance with claim 1, wherein said subject is human.

34. A method in accordance with claim 1, wherein said compound is selected from the group consisting of

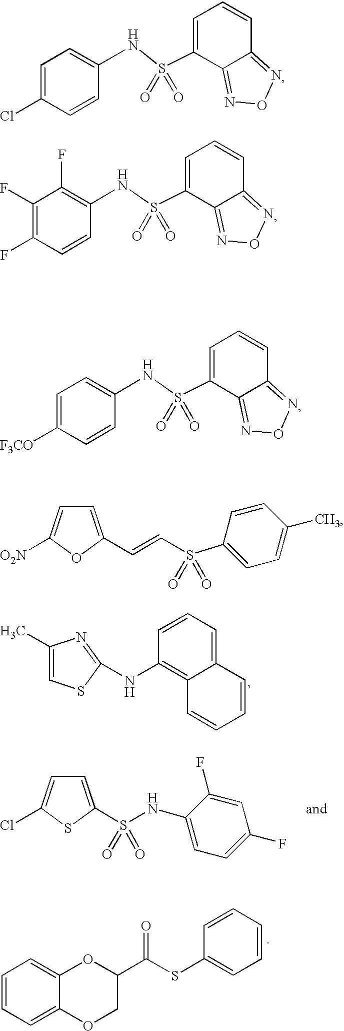

35. A method in accordance with claim 1, wherein said CCR4-mediated disease or condition is selected from the group consisting of multiple sclerosis, rheumatoid arthritis, type I diabetes, psoriasis, cancer and HIV infection; Ar¹ is a substituted heterocycle; X is —SO₂NH—; and Ar² is a substituted phenyl.

36. A method in accordance with claim 1, wherein said CCR4-mediated disease or condition is selected from the group consisting of multiple sclerosis, rheumatoid arthritis, type I diabetes, psoriasis, cancer and HIV infection; Ar¹ is a substituted heterocycle; X is —NH—; and Ar² is naphthyl.

37. A pharmaceutical composition for the treatment of a CCR4-mediated disease or condition, said composition comprising a pharmaceutically acceptable carrier and an effective amount of a compound which inhibits the binding of MDC or TARC to CCR4, said compound having the formula:
Ar¹—X—Ar² (I)
Ar¹ and Ar² are each members independently selected from the group consisting of substituted or unsubstituted aryl, substituted or unsubstituted fused aryl-heterocyclic ring systems and substituted or unsubstituted heteroaryl; and
X is a linking group selected from the group consisting of —N(R)—, —C(O)S—, —CH═CHSO₂— and —SO₂N(R)— wherein R is a member selected from the group consisting of H and substituted or unsubstituted (C₁-C₈)alkyl.

38. A composition of claim 37, wherein X is —NH—.

39. A composition of claim 37, wherein X is —SO₂NH—.

40. A composition of claim 37, wherein Ar¹ and Ar² are each substituted or unsubstituted members independently selected from the group consisting of:

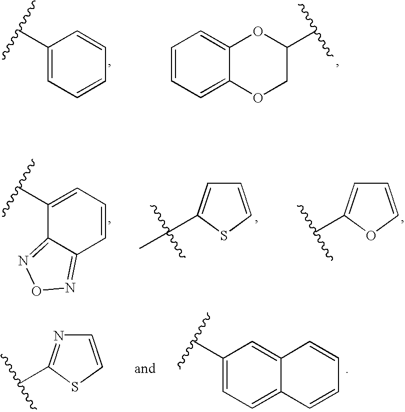

41. A composition of claim 37, wherein Ar¹ is substituted heteroaryl and Ar² is substituted or unsubstituted aryl.

42. A composition of claim 41, wherein said Ar¹ is a substituted heteroaryl selected from the group consisting of substituted thiazolyl, substituted thienyl, and substituted furanyl.

43. A composition of claim 41, wherein said Ar² is a substituted or unsubstituted phenyl or a substituted or unsubstituted naphthyl.

44. A composition of claim 41, wherein Ar¹ is a phenyl group having from 1 to 4 substituents independently selected from the group consisting of halogen, hydroxy, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino.

45. A composition of claim 44, wherein said phenyl group has from 1 to 3 substituents independently selected from the group consisting of halogen, (C₁-C₄)haloalkyl, (C₁-C₄)haloalkoxy, nitro, cyano, and (C₁-C₄)acyl.

46. A composition of claim 37, wherein Ar¹ is a substituted or unsubstituted monocyclic or bicyclic heterocycle.

47. A composition of claim 46, wherein said heterocycle is selected from the group consisting of pyrrolyl, pyrazolyl, imidazolyl, pyrazinyl, oxazolyl, isoxazolyl, thiazolyl, furyl, thienyl, pyridyl, pyrimidyl, benzothiazolyl, benzoxadiazolyl, purinyl, benzimidazolyl, indolyl, isoquinolyl, quinoxalinyl and quinolyl.

48. A composition of claim 47, wherein said heterocycle is selected from the group consisting of thienyl, thiazolyl and benzoxadiazolyl.

49. A composition of claim 37, wherein said compound is selected from the group consisting of

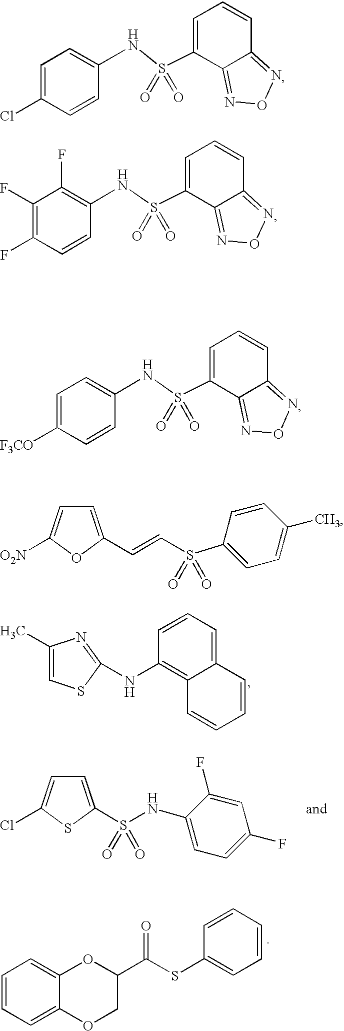

50. A method for modulating CCR4 function in a cell, comprising contacting said cell with a CCR4-modulating amount of a composition of claim 37.

51. A method for modulating CCR4 function, in which said cell is contacted with a CCR4 protein with a therapeutically effective amount of the composition of claim 37.
